(19) United States
(12) Patent Application Publication
(10) Pub. No.: US 2014/0324403 A1
(43) Pub. Date: Oct. 30, 2014

(54) DETERMINING A RANGE OF MOTION OF AN ANATOMICAL JOINT

(71) Applicant: Brainlab AG, Feldkirchen (DE)

(72) Inventor: Hubert Gotte, Muenchen (DE)

(21) Appl. No.: 14/363,347

(22) PCT Filed: Dec. 9, 2011
(86) PCT No.: PCT/EP2011/072324

§ 371 Date: Jun. 6, 2014

(51) Int. Cl. A61F 2/46; G06F 19/12; A61B 19/00

(52) U.S. Cl. CPC A61F 2/4657; A61B 19/50; A61B 19/5225; A61B 19/5244; G06F 19/12; A61B 2019/502; A61B 2019/505; A61B 2019/5268; A61F 2002/4668
USPC 703/2

(57) ABSTRACT

A data processing method for determining a position of at least two body parts relative to one another, which at least two body parts are connected to one another by at least one joint, the method being executed by a computer and comprising acquiring initial body part position transformation data comprising initial body part position transformation information describing an initial transformation between positions of the at least two body parts in a predetermined pose of the at least two body parts; acquiring alteration transformation data comprising alteration transformation information describing a transformation between a position of a first alteration part of at least one first body part of the at least two body parts, which first alteration part represents a first virtual anatomical alteration to the at least one first body part, and a position of an anatomically unaltered part of the at least one first body part; determining, based on the initial body part position transformation data and the alteration transformation data, altered body part position transformation data comprising altered body part position transformation information describing a transformation between a position of the first alteration part and a position of a second other one of the at least two body parts in the predetermined pose.

The present invention is directed to a method for determining a position of body parts relative to one another in accordance with claim 1 and a corresponding computer program and computer running that program.

In joint surgery, in particular knee surgery, it is desirable to judge an influence of a surgical procedure on the range of motion of the respective joint. Presently, such an influence can only be determined by manipulating the joint after surgery has commenced. This, however, means that a surgical error may already have occurred when an undesired influence on the range of motion is determined Generally, such influences may not be reversed and the patient may then suffer from side effects of the operation such as an undesirably limited range of motion of the joint.

A problem to be solved by the invention thus is to improve the reliability of determining in particular pre-operatively the influence of a surgical procedure on the range of motion which a joint will have.

This problem is solved by the subject-matter of any appended independent claim. Advantages, advantageous features, advantageous embodiments and advantageous aspects of the present invention are disclosed in the following and contained in the subject-matter of the dependent claims. Different advantageous features can be combined in accordance with the invention as long as technically sensible and feasible. In particular, a feature of one embodiment which has the same or similar function of another feature of another embodiment can be exchanged. In particular, a feature of one embodiment which supplements a further function to another embodiment can be added to the other embodiment.

The inventive method is directed to a data processing method for determining a position of at least two body parts relative to one another, which at least two body parts are preferably connected to one another by in particular at least one joint. In the framework of this disclosure a position is defined by specific coordinates in a reference system (in particular, a coordinate system). Preferably, the entity of which the position is described (the entity being e.g. a physical structure such as a body part or a medical instrument or a virtual structure such as a point defined in space) rests relative to that coordinate system, in particular the coordinate system is centered (i.e. has its origin) in the respective entity. Preferably, the coordinate systems in which the positions of different body parts are defined, are different from one another. In particular, the bases of the coordinate systems are not identical. As becomes clear from the above, the position of the entity, in particular body part, is defined as an absolute position only with respect to its assigned reference system (in particular, coordinate system). Where in the framework of this disclosure a position of an entity (e.g. a body part or instrument) relative to another entity (e.g. another body part or another instrument) shall be determined in a coordinate system which is different from its assigned coordinate system, this is explicitly stated and the position denoted as a relative position or a position of the one entity relative to the other entity.

Preferably, the body parts of which the position relative to one another is to be determined are connected to one another by in particular at least one joint (in particular natural or artificial, in the latter case in particular implanted, anatomical joint). This connection may be established by direct physical contact between the body parts (e.g. by touching each other) and/or by indirect physical contact such as via ligaments or cartilage. An example of a joint having both indirect and direct physical contact between the body parts forming that joint is the knee in which connecting structures, in particular ligaments (e.g. cruciate ligaments) and/or cartilage (e.g. the meniscus), are present and the proximal end of the tibia and the distal end of the femur have direct physical contact to one another (i.e. touch one another).

Preferably, initial body part position transformation data is acquired which comprises initial body part position transformation information. The initial body part position transformation information describes in particular an initial transformation between positions of the at least two body parts in a predetermined pose of the at least two body parts. A pose can be represented mathematically by a transformation. Within the framework of this disclosure, transformations such as e.g. the initial transformation in particular are coordinate transformations which are represented by preferably a linear transformation. Such a linear transformation is preferably embodied by a matrix which is preferably invertible and transforms a position in a first coordinate system, e.g. the position of a first body part of the at least two body parts in a first body part coordinate system (resting relative to the first body part), into a position in a second other (in particular different) coordinate system, e.g. the position of a second one of the at least two body parts in a second body part coordinate system (resting relative to the second body part), in particular maps the positions between the coordinate systems. To this end, the transformations, e.g. the initial transformation, may comprise or consist of a basis transformation, in particular a transformation into a common basis the coordinate system, e.g. of the first body part coordinate system and the second body part coordinate system in which the position of the first body part and the position of the second body part can be defined relative to a common origin. The initial transformation is preferably acquired for a predetermined functional pose of the body parts. A functional pose of the body parts encompasses both the position of the two body parts relative to one another and their orientation in particular relative to one another or to a common reference entity (e.g. a physical structure such as an instrument, or virtual structure such as a predetermined position). The orientation (in particular, three-dimensional orientation) is in particular defined by an angle which is enclosed by the two body parts at the location of the joint (in particular, a flexion angle in a specific functional movement state of the joint and/or the body parts connected by the joint—the pose may therefore also be called functional pose). Preferably, three such angles in the basic movement directions of the joint which characterizes the range of motion are used. In the case of the joint being a knee, these angles preferably are the internal/external rotation angle, the varus/valgus angle and the flexion/extension angle. For other joints in the human body, such as a finger joint or an ankle joint, corresponding angular directions of movement may be defined. The position of the body part relative to one another is furthermore defined by translations of the body parts relative to one another, in the case of the joint being a knee by shifts in the mediolateral, anterioposterior and proximodistal direction. Corresponding shifts may be defined for other joints in the human body such as a finger joint or an ankle joint.

The initial body part position transformation data is preferably acquired by a navigation system. For example, the body parts may be provided with marker devices such as retroreflective markers which are configured to reflect infrared electromagnetic radiation which is emitted from e.g. a source device of a navigation system and detected e.g. by an infrared camera. Other tracking technologies suited well beside optical tracking are electromagnetic or ultrasound or mechanical tracking. The body parts are then brought into the predetermined functional pose and the position of the markers in particular in a coordinate system which is specific for the needs of the navigation system are determined. Preferably, a plurality of such measurements are conducted and position parameters are extracted from the measured data which describe the pose of the joint components (i.e. the body parts) in a specific movement state, in particular in a specific rotational angle, in particular a specific three-dimensional rotational angle. From these position parameters, the initial transformation can be determined by way of known linear algebra and provided to the inventive method.

Preferably, alteration transformation data comprising alteration transformation information is acquired. The alteration transformation information in particular describes a transformation (in particular, coordinate transformation) between a position of a first alteration part of at least one first body part of the at least two body parts and a position of an anatomically unaltered part of the at least one first body part. This transformation is in the framework of this disclosure also called alteration transformation. This first alteration part preferably represents, in particular comprises or is (in particular, is the result of), a virtual anatomical alteration (in particular first virtual anatomical alteration) to the at least one first body part. After the change has been implemented, the changed part of the body part including any possibly inserted (implanted) implant is also termed alteration part. The virtual anatomical alteration is virtual, i.e. preferably implemented in particular conducted or embodied, by means of data processing, in particular conducted by a computer or embodied by a program. The virtual anatomic alteration is further preferably conducted pre-operatively, i.e. before any invasive procedure is conducted on the patient to whom the body parts belong. Even further, the virtual anatomical alteration is conducted independently of whether such an invasive procedure is conducted or not or envisaged or not. In particular, the virtual anatomical alteration is performed by means of image processing and preferably includes editing of medical image data such that parts of an image which represent the first (or second) alteration part are changed (in particular, regarding their virtual physical constituents and anatomy, in particular geometry), in particular by removing part of the graphical representation of the changed body part and/or by inserting a graphical representation of an implant (in particular a surgical implant) at the location of the changed part of the body part. It therefore is clear that conducting the virtual anatomical alteration merely relates to steps of data processing, in particular of simulating a physical change to at least the part of the one body part, and does not comprise or encompass an invasive step representing a substantial physical intervention on the body which requires professional medical expertise to be carried out and which entails a substantial health risk even when carried out with the required professional care and expertise. The alteration transformation information in particular describes a transformation between a position of that first alteration part and a position of an anatomically unaltered part of the in particular same at least one body part. The term of transformation is again to be understood in analogy to the definition presented for the initial body part position transformation information. For example, the alteration transformation represents a mapping between a position of the first alteration part and the position of an unaltered part of the at least one body part. More specifically, the alteration transformation may define a mapping between a femur-side implant (in particular, surgical implant) for a total endoprosthesis (TEP) of the knee and an unaltered part of the femur bone on which in particular no surgery is carried out, i.e. on which no anatomical changes will be or are being implemented (simulated) when implementing the virtual anatomical alteration. An example of such an unaltered part of the femur would be a part of the femur shaft or the femur head.

Preferably and according to a more specific embodiment, the alteration transformation information further describes a transformation (in particular, coordinate transformation) between a position of a second alteration part of a second, other one of the at least two body parts and a position of an anatomically unaltered part of the second body part to which the second alteration part belongs. The second alteration part represents, in particular comprises or is (in particular, is the result of), a virtual anatomical alteration (this term being defined in analogy to the above regarding the first virtual anatomical alteration), In that case, the alteration transformation information preferably further describes a transformation between the position of the first alteration part (of the first body part) and the position of the second alteration part (of the second body part). It is to be noted that the first and second body part are preferably different from one another, for example the second body part may be a tibia and the virtual anatomical alteration carried out on it may be to replace the tibia head by a tibia-side implant for a total endoprosthesis (TEP) of the knee. Preferably, the first and second altered part are parts of the body parts which are located opposite to one another in the joint and in particular are designed to have (direct or indirect) physical contact with one another. Such a physical contact shall also be established after carrying out the virtual anatomical alteration, for example the femur-side implant and the tibia-side implant are designed to preferably directly contact one another after carrying out the envisaged surgery. Such a direct physical contact is preferably also simulated in the framework of the inventive method.

The second virtual anatomical alteration also is virtual, this term being defined as for the first virtual anatomical alteration. The first and second virtual anatomical alterations preferably describe alterations which are different in particular regarding their anatomical and geometrical properties (in particular, their anatomical and geometrical changes). Therefore, also the first and second alteration parts preferably are different from one another in regard of at least these properties.

The alteration transformation data is acquired preferably based on, in particular from, a data set (also called “surgical plan”) comprising predetermined information about the position of the first alteration part relative to the anatomically unaltered part of the at least one first body part and, if applicable, the position of the second alteration part relative to an unaltered part of the second body part.

Preferably, altered body position transformation data is determined based on the initial body part position transformation data, in particular based on the initial body part transformation information, and based on the alteration transformation data, in particular based on the alteration transformation information.

The altered body part position transformation data in particular comprises altered body part position transformation information which describes a transformation between a position of the first alteration part and a position of the second body part in the predetermined pose, in particular after the first virtual anatomic alteration (i.e. after the first virtual anatomical alteration has been conducted). The second body part may also include a second alteration part which represents a second virtual anatomical alteration as described above. However, the second body part may also be anatomically unaltered in its entirety. In case the second body part includes the second alteration part, the altered body part transformation information preferably describes a transformation between a position of the first alteration part and a position of (only) the second alteration part.

In the case of a virtual anatomical alteration to the first alteration part (part of the first body part), the virtual anatomical alteration is called a first virtual anatomical alteration. In the case of a virtual anatomical alteration to the second alteration part (part of the second body part), the virtual anatomical alteration is called a second virtual anatomical alteration.

In the framework of this disclosure, the term of pre-operative state denotes a state of the first body part and/or the second body part (more generally, of the joint which connects the first and second body parts) before the first virtual anatomical alteration and/or the second virtual anatomical alteration, respectively, is conducted (in particular, by executing the corresponding program steps on a computer). In analogy, the term of post-operative state denotes a state of the first body part and/or the second body part after the first virtual anatomical alteration and/or the second virtual anatomical alteration, respectively, has been conducted (in particular, by executing the corresponding program steps on a computer). Thus, the post-operative state actually is a virtual post-operative state. The post-operative state in particular defines a state of the first body part and/or the second body part (more generally, of the joint which connects the first and second body parts) which in the framework of the inventive method is simulated based on the alteration transformation data, in particular based on the alteration transformation information, more particularly based on information about the first and/or second virtual anatomical alteration.

Preferably, contact position data is determined based on the altered body part position transformation data, in particular based on the altered body part position transformation information. The contact position information describes in particular the positions of the at least two body parts in which at least two of those at least two body parts are considered to have in particular a predetermined, more particularly a desired, (direct or indirect) physical contact with one another. In the framework of this disclosure, the predetermined (desired) contact describes in particular a relative position of at least two physical structures with regard to each other in which the physical structures touch each other but do not intersect each other in two- or three-dimensional space. More particularly, the contact position information describes positions (contact positions) of the at least two body parts in which at least one alteration part has such physical contact with at least one other of the at least two body parts. Even more particularly, the contact position information describes positions of the at least two body parts in which at least two alteration parts (in particular, the first and the second alteration part) of the at least two body parts have such physical contact with one another.

Alternatively or additionally, the contact position information may also describe positions of the at least two body parts in which at least one alteration part and at least one anatomically unaltered part of at least two of the at least two body parts have such contact with one another. The contact positions in particular are such positions in which a predetermined state of contact is established.

Preferably, the contact position data is determined by determining a position of the at least two body parts relative to one another (or the alteration part and the other body part relative to one another, respectively) in which the respective physical contact between the at least two body parts (or their constituents such as alteration parts) is established. The step of determining the contact position data again relies purely on steps of data processing and does not involve any surgical activity, in particular no invasive procedure.

The contact position information is preferably determined by a method of determining contact position parameters of a joint connecting two bones as disclosed in the applicant's co-pending European patent application having the title “Method of determining contact position parameters of a joint connecting two bones” and the attorney's reference 58420 XX which was filed on the same day. The contact position data is preferably determined based on acquiring a data set comprising six parameters describing a contact position, acquiring a subset of n of the parameters as an input parameter data set, n being an integer in the range of 1 to 5 describing the number of predetermined parameters, and determining 6-n free parameters of the contact position. Preferably, a resulting pose of the first and second body parts is determined based on the contact position data,

Preferably, the contact position data is determined based on acquiring a data set comprising six parameters describing altered body transformation information, acquiring a subset of n of the parameters in the data set as an input parameter data set, n being an integer in the range of 1 to 5 (preferably, n=2) describing the number of predetermined parameters determining 6-n free parameters of the altered body part transformation information and changing the altered body transformation information according to the determined 6-n free parameters, and wherein a resulting pose of the first and second body parts is determined based on the contact position data, which contact data is determined based on the changed altered body transformation data.

The method of determining contact position parameters of a joint connecting two bones is also part of this disclosure and particular features of this method are described in the following. The features of the method of determining contact position parameters of a joint connecting two bones may be combined with the features of the aforementioned method without prejudice.

In the human anatomy, two bones are typically connected via a joint which exhibits certain kinematic properties. The kinematic properties define a range of motion between the two bones. In other words, the joint typically supports a range of relative positions between the two bones. Such a relative position usually is a contact position in which the surfaces of the two bones are in contact with each other. In particular, there are two or three points of contact or a line contact between the two bones. In general, each contact position is defined by six parameters, wherein three parameters define a translational shift in three dimensions which are typically pairwise orthogonal. In a preferred example, the directions of the shifts are the proximodistal (pd) direction, the anterioposterior (ap) direction and the mediolateral (ml) direction. The other three parameters define a rotational shift or angular alignment, preferably about three pairwise orthogonal axes. Preferably, the three rotational parameters relate to the flexion/extension angle (fe), the internal/external angle (ie) and the varus/valgus angle (vv). In particular, the six parameters of a contact position describe the relative position between a first coordinate system of the first bone and a second coordinate system of the second bone.

There are certain desired applications which require the six parameters of a contact position when a subset of the parameters of the contact position is given. An exemplary application is the visualization of the range of motion of the joint. Therefore, a need arises to provide a method for determining the six parameters of a contact position of a joint which connects two bones, wherein the method in particular should be computationally effective in order to allow a real-time or on-the-fly determination.

The method of determining contact position parameters of a joint connecting two bones thus relates to a data processing method for determining six parameters of a contact position of a joint which connects two bones. One step of the method is acquiring a plurality of sample contact position datasets, wherein each dataset comprises six parameters. These six parameters of the sample contact position datasets correspond to the six parameters describing the contact position. The sample contact position datasets, also referred to as node datasets, represent known contact positions of the two bones via the joint.

The method of determining contact position parameters of a joint connecting two bones further comprises the step of acquiring a subset of n of the parameters of the contact position as an input parameter dataset. In other words, n of the parameters of the contact position are predetermined, while the remaining (free) parameters of the contact position have to be determined. The variable n is an integer in the range of 1 to 5, preferably in the range of 3 to 5, and more preferably n has the value of 4.

Other steps of the method of determining contact position parameters of a joint connecting two bones are selecting at least two of the sample contact position datasets or of a subset of the sample contact position datasets based on the input parameter dataset and determining the m=6-n remaining parameters of the contact position based on the at least two selected sample contact position datasets. In other words, the input parameter dataset is used to select two or more appropriate sample contact position datasets from which the remaining parameters of the contact position are calculated.

Such a model of contact position parameters is also termed “continuous joint model (CJM)”.

With this approach, a database of sample contact position datasets representing discrete sample contact positions of the joint is used as a basis for calculating the six parameters of any (intermediate) contact position. Since the method of determining contact position parameters of a joint connecting two bones does not use a computationally extensive model, such as a collision detection model, to determine the remaining parameters, but approximates the remaining parameters from (surrounding) sample contact positions, the determination is considerably fast.

Preferably, the remaining parameters are determined by interpolation or extrapolation. In particular, interpolation is preferably applied if all n parameters of the input parameter dataset are lying between the corresponding n parameters of the selected sample contact position datasets. Extrapolation is preferably applied if at least one of the n parameters of the input parameter dataset is lying beyond the corresponding parameters of the selected sample contact position datasets.

The selected sample contact position datasets must be suitable for determining the remaining parameters of the contact position. Preferably, the selected sample contact position datasets correspond to sample contact positions which are, regarding the n input parameters, the nearest neighbours of the contact position. In other words, the criterion for selecting a sample contact position dataset is the distance between the n parameters of the input parameter dataset and the corresponding parameters of a sample contact position dataset. Nearest neighbours of the contact position thus are sample contact positions with a minimum distance to the contact position, wherein the distance is defined by the n input parameters and the corresponding parameters of a sample contact position. A maximum distance between the input parameters of the contact position and the corresponding parameters of the sample contact positions can be defined, wherein sample contact positions with a larger distance cannot be selected. Further, a minimum number of sample contact positions to be selected can be defined.

In one embodiment, the distance between the contact position and a sample contact position is calculated using a Minkowski distance function. For n parameters, the p-norm distance is therefore given by the expression

$${( {\sum }_{i = 1}^{n} {\vert {x}_{i} - {y}_{i} \vert}^{p} )}^{1 / p}$$

with p≧1. The variable x represents the contact position, the variable y represents a sample contact position and i is an index for the input parameters.

In general, any suitable approach can be used for the interpolation or the extrapolation. However, preferred types of interpolation are a spline interpolation or an interpolation using inverse distance weighting. Spline interpolation in particular refers to a third degree spline interpolation, but also encompasses any other grades as well as B-splines or Bezier curves.

Inverse distance weighting is simple in implementation and can easily be applied to irregularly distributed data. An example for inverse distance weighting is given by the formula

$$u ( x ) = {\sum }_{i = 0}^{S} \frac{{w}_{i} ( x ) {u}_{i}}{{\sum }_{j = 0}^{S} {w}_{j} ( x )}$$

$$with$$

$${w}_{i} ( x ) = \frac{1}{{d ( x , {x}_{i} )}^{p}} .$$

In this formula, u is the parameter value to be determined at the contact position x. It is calculated based on S neighbours. The values of the corresponding parameter in the selected S neighbours are given by uᵢ with i running from 1 to S. The variable d describes the distance, regarding the n input parameters, between a neighbour xᵢ and the contact position x. The variable w is a weighting factor or weight which depends on the distance d. The parameter p is a positive real number shaping the interpolation characteristics.

In one embodiment, the sample contact position datasets are arranged in an n-dimensional array and each array entry comprises the m remaining parameters. With this organizational data structure, the selection of the at least two sample contact position datasets is easy and computationally effective, in particular if the sample contact positions are arranged at equidistant intervals. This means that the values of one particular parameter of the sample contact position, and in particular of all n parameters of the sample contact position which are used as input parameters, can assume discrete values, wherein the discrete values have an equidistant distance.

With sample contact positions at equidistant intervals, the selection of two or more sample contact position datasets is computationally effective, in particular if the index in a dimension of the array corresponds to a multiple of the equidistant interval. If, for example, the interval for an angle is 5 degrees, then an index of 0 corresponds to 0 degrees, an index of 1 corresponds to 5 degrees, an index of 2 corresponds to 10 degrees, and so on. As an option, an offset is added to the index in order to represent shifts symmetrically arranged around a zero shift. So if an angle as one parameter of the contact position is given in 5 degree increments and the index i in the corresponding dimension of the array runs from 0 to 71, then the angle corresponding to an index i is given by i×5 degrees-175 degrees. In a reverse manner, the array index of a sample contact position can be calculated from a particular angle.

In another embodiment, the sample contact position datasets are stored as lists of six parameters each. For example, the six parameters are consecutively written in a line of a text and each line of the text constitutes a sample contact position dataset. With this approach, the parameters of the contact position which form the input parameters can easily be adapted to the desired application in which the method of determining contact position parameters of a joint connecting two bones is used. For example, in one application the remaining parameters are two angles, such as flexion/extension and varus/valgus, while in another application the remaining parameters are all translational shift parameters. Of course, any combination of translational and rotational parameters can constitute the remaining parameters.

In one embodiment, a sample contact position dataset is void for an impossible joint contact position. An impossible joint contact position is a contact position which cannot be assumed by the joint. For example, a sample contact position dataset can be made void by assigning a particular value to one of the parameters. In one exemplary implementation, a value outside of the possible range of 360 degrees is assigned to a parameters corresponding to an angle. Since an angle can only lie within a 360 degree range, a value outside this range can be used to indicate a void dataset.

In another embodiment, a sample contact position dataset further comprises affiliate information, also referred to as label, which indicates that the sample contact position belongs to a contact profile of contact positions. A contact profile of contact positions represents for example a particular movement, such as for walking or standing up regarding a knee joint. A contact profile consists of a sequence of contact positions. Preferably, an additional parameter further defines the position of the sample contact position within the sequence of contact positions, i.e. the sequence number.

The labels enable filtering of the sample contact position datasets. In one embodiment, neighbours for interpolating or extrapolating are only selected from sample contact position datasets which have a predetermined label, in particular a label corresponding to a contact profile. Sample contact positions with a particular label may be used as nodes for finer subdivision of the path between them.

Affiliate information can further comprise a 4×4 matrix which describes the spatial transformation from one bone, or implant, to the other(s).

In another embodiment, the step of determining the m=6−n remaining parameters of the contact position is repeated for a sequence of input parameter datasets, which is also referred to as a parameter profile, thus resulting in a sequence of contact positions. The parameter profile preferably corresponds to a contact profile, which means that it might represent a particular movement. With this embodiment, the properties of the joint can be determined for a particular sequence of input parameters for analysis. For example, the properties of the joint can be displayed graphically, for example by plotting the remaining parameters as curves over the variation of the input parameters. In addition, the bones connected by the joint can be displayed in an animation representing the sequence of input parameter datasets.

One aspect of the method of determining contact position parameters of a joint connecting two bones relates to the acquisition of a sample contact position dataset. In one embodiment, a sample contact position dataset is determined by virtually positioning three-dimensional images of the two bones in a computer such that they are in contact. This might be done using a CAD software provided with a 3D representation of the bones or at least the parts of the bones which form the joint. In the software, the relative position of the two bones is modified until they are in contact. This relative position of the bones thus positioned is then used as a sample contact position. In a particular embodiment, the relative position between the two bones is adjusted manually, for example using an input device such as a mouse, a joystick, a trackball, a pointer or a touch screen. The capabilities of the CAD software can be used in order to determine whether or not the two bones are in contact. The software can for example display a cross-sectional view or a perspective which could not be assumed in the real world, for example from within one of the bones or a cavity of one of the bones.

As an option, a sample position dataset is automatically determined by using collision detection of three-dimensional models of the two bones.

As an alternative, a sample contact position dataset is determined by measuring a real joint. A real joint is physiological joint, for example formed by the two bones or two components of an implant which form an artificial point.

The further features of the inventive method disclosed in the following may be combined with the aforementioned features of the inventive method and/or the method of determining contact position parameters of a joint connecting two bones without prejudice. However, the features described in the following may also be considered to form a method of its own which in particular does not require the aforementioned features for its implementation.

Preferably, a plurality of positions of at least two of the at least two body parts relative to one another in different functional poses is determined based also on the altered body part position transformation data, in particular based on the altered body part position transformation information. In particular, the relative positions of at least two of the at least two body parts relative to one another are calculated in a multi-dimensional space which comprises in general six dimensions.

The coordinate transformation described by the altered body part position transformation is applied to calculate the relative positions of the at least two body part relative to one another, in particular for determining a relative position and/or coordinate transformation between the assigned coordinate systems of the first alteration part and the second alteration part (i.e. the first alteration part coordinate system and the second alteration part coordinate system). By doing so, a correct positioning of the first alteration part and the second alteration part relative to one another is supported. In particular a suitable physical contact between the two parts is established according to the aforementioned method of determining contact position parameters of a joint connecting two bones. Thus, the two body parts including their respective alteration parts (as far as present) preferably undergo simulation of a plurality of movement states, in particular of functional poses or states of continuous movements, and their relative position is determined for each of the movement states. Preferably, a graphical output is generated based on the results of the simulation, in particular based on changes to the data set comprising the predetermined information about the position of the first alteration part relative to the first body part and, if applicable, the second alteration part relative to the second body part. The graphical output is updated based on the altered body part position transformation data. Such a procedure provides the specific advantage that an update of the alteration transformation data may be conducted without having to again acquire the initial body part position transformation data. Thereby, the expected range of motion of an anatomical joint in the post-operative state may be determined based on the range of motion of the pre-operative state.

This procedure supports determining the range of motion of the joint which connects the at least two body parts with one another in consideration of the surgical procedure which is envisaged, in particular in a post-operative state. In particular, the range of motion may be determined before surgery commences without having to conduct any invasive (surgical) procedure on the patient's body.

Preferably, joint contact enforced deviation data comprising joint contact enforced deviation information is acquired. The joint contact enforced deviation information in particular describes a predetermined relationship between a movement state, in particular a pose or continuous movement, of the at least two body parts and a tension attribute or qualifier of the joint. The tension attribute of the joint is described by in particular at least one of (mechanical) stress and laxity of the joint.

Preferably, expected contact enforced deviation data is determined based on the altered body part position transformation data. The expected contact enforced deviation data comprises a predetermined relationship between an expected change of movement state, in particular an expected change of pose, of the at least two body parts due to the altered body transformation and a tension attribute, in particular at least one of stress and laxity, of the joint.

Preferably, determining the expected contact enforced deviation data comprises determining a distance of predetermined locations in or on at least one of the at least two body parts from a predetermined position, in particular a geometric feature (such as a plane or line), before implementing (in particular, conducting) the first virtual anatomic alteration to a distance of the predetermined locations from the predetermined position after implementing (in particular, conducting) the first virtual anatomic alteration. Preferably, the expected contact enforced deviation data is determined for a plurality of movement states in the post-operative state. This supports determining the mechanical stability and mechanically possible movement states (and therefore the range of motion) of the joint in the post-operative state. The predetermined locations preferably are locations at which a connecting structure such as a ligament is attached to the at least one of the at least two body parts, for example the epicondyle points of the knee in case the joint is or represents a knee. The predetermined position, from which the distance of the predetermined location is measured, may be a specific anatomical location, in particular a location on another one of the at least two body parts (preferably, the second body part), or a virtual reference location such as a predetermined line or plane or singular point in space which preferably has a fixed position relative to the second body part. In the case of the joint being a knee, a distance of the medial epicondyle point from the predetermined position and a distance of the lateral epicondyle point from the predetermined position is determined in the pre-operative state of the at least two body parts and in the post-operative state. Then, each post-operative distance is compared to the respective pre-operative distance. Comparing the pre-operative and post-operative distances preferably comprises determining a difference between the distances, in particular subtracting the length of the pre-operative distance from the length of the post-operative distance (or vice versa). This comparison then preferably forms a basis for determining the post-operative, in particular changed, tension in the connecting structures (e.g. in the medial and lateral collateral ligaments).

The predetermined geometric feature preferably is a reference line or reference plane having preferably a fixed position relative to the second body part and being in particular defined in the coordinate system assigned to the second body part. The predetermined position preferably is common for determining the distances for all of the predetermined locations, i.e. it is assigned commonly to all of the predetermined locations. Further preferably, the common reference line or reference plane is parallel to the transversal plane (and/or the transversal or mediolateral axis) of a body, in particular patient's body, of which the at least two body parts are part of.

Preferably, the joint connecting the at least two body parts to one another is a knee joint. Further preferably, an angle, in particular a varus and/or a valgus angle between straight lines connecting the predetermined locations in the pre-operative state and the post-operative state is determined based on the comparison of the distances. This angle in particular comprises a change to the varus/valgus angle in the pre-operative state.

The direction of the straight line connecting the predetermined locations is preferably determined for the pre-operative state from the aforementioned medical image data and for the post-operative state from the simulation of the joint geometry which is in particular based on the altered body part position transformation data, in particular the altered body part position transformation information.

Preferably, the method in accordance with the present disclosure comprises plotting a diagram of the expected contact enforced deviation information as a function of movement state, in particular of pose (more particularly, as a function of flexion angle and/or as function of internal/external rotation and/or as a function of mediolateral shift and/or as a function of anterioposterior shift and/or as a function of varus/valgus angle and/or as a function of proximodistal shift), of the joint. In such a diagram, regions of comparably decreased or increased varus or valgus angle of the at least two body parts relative to one another (or of the joint, in particular knee joint, respectively) are highlighted. The information about the location of such regions of angular orientation in certain intervals of flexion angles is preferably determined based on information about the varus/valgus orientation of the at least two body parts in dependence on the contact enforced deviation information. Such a graphical display supports surgical planning which is directed to avoiding increased or decreased ligament tension in the post-operative state which may lead to ligament contractions and difficulties in patient rehabilitation.

Preferably, the disclosed method comprises graphically displaying graphical representations of the at least two body parts in particular in a plurality of movement states (in particular in a plurality of poses), after the first virtual anatomical alteration has been implemented. In particular, the graphical display may also be conducted after the second virtual anatomical alteration has been implemented. This provides a graphical impression to a user e.g. a surgeon or physiotherapist, about the geometry of the at least two body parts (and therefore the joint) in the post-operative state for a plurality of movement states, in particular a plurality of poses which differ in particular by the flexion angle of the joint.

Where in the framework of this disclosure it is mentioned that data comprises certain information, the respective data in particular is or consists of the respective information. Where in the framework of this disclosure it is mentioned that information describes a specific information content, the information may also be considered to be or consist of the respective information content.

Within the framework of this disclosure, the term of range of motion describes the freedom of the joint connecting the at least two body parts to move. In particular, such a freedom to move is defined by a set of movement states of the joint in which there is no mechanical blockage or interlock of the joint which prohibits the joint from taking on the respective movement state. In particular, the range of motion constitutes a set of all the possible movement states of the joint, in particular such movement states which are possible in view of mechanical limitations.

The method in accordance with the invention is in particular a data processing method. The data processing method is preferably performed using technical means, in particular a computer. In particular, the data processing method is executed by or on the computer. The computer in particular comprises a processor and a memory in order to process the data, in particular electronically and/or optically. The calculating steps described are in particular performed by a computer. Determining or calculating steps are in particular steps of determining data within the framework of the technical data processing method, in particular within the framework of a program. A computer is in particular any kind of data processing device, in particular electronic data processing device. A computer can be a device which is generally thought of as such, for example desktop PCs, notebooks, netbooks, etc., but can also be any programmable apparatus, such as for example a mobile phone or an embedded processor. A computer can in particular comprise a system (network) of “sub-computers”, wherein each sub-computer represents a computer in its own right. The term of computer encompasses a cloud computer, in particular a cloud server. The term of cloud computer encompasses cloud computer system in particular comprises a system of at least one cloud computer, in particular plural operatively interconnected cloud computers such as a server farm. Preferably, the cloud computer is connected to a wide area network such as the world wide web (WWW). Such a cloud computer is located in a so-called cloud of computers which are all connected to the world wide web. Such an infrastructure is used for cloud computing which describes computation, software, data access and storage services that do not require end-user knowledge of physical location and configuration of the computer that delivers a specific service. In particular, the term “cloud” is used as a metaphor for the internet (world wide web). In particular, the cloud provides computing infrastructure as a service (IaaS). The cloud computer may function as a virtual host for an operating system and/or data processing application which is used for executing the inventive method. Preferably, the cloud computer is an elastic compute cloud (EC2) provided by Amazon Web Services™. A computer in particular comprises interfaces in order to receive or output data and/or perform an analogue-to-digital conversion. The data are in particular data which represent physical properties and/or are generated from technical signals. The technical signals are in particular generated by means of (technical) detection devices (such as for example devices for detecting marker devices) and/or (technical) analytical devices (such as for example devices for performing imaging methods), wherein the technical signals are in particular electrical or optical signals. The technical signals represent in particular the data received or outputted by the computer.

The invention also relates to a program which, when running on a computer or when loaded onto a computer, causes the computer to perform one or more or all of the method steps described herein and/or to a program storage medium on which the program is stored (in particular in a non-transitory form) and/or to a computer on which the program is running or into the memory of which the program is loaded and/or to a signal wave, in particular a digital signal wave, carrying information which represents the program, in particular the aforementioned program, which in particular comprises code means which are adapted to perform any or all of the method steps described herein.

The invention also relates to a navigation system for computer-assisted surgery, comprising:

a computer, in particular the computer defined in the preceding paragraph;

a detection device for detecting the position of the first and second body parts (in particular by detecting markers attached to them) and to supply information about the position to the computer;

a data interface for receiving the position information from the detection device and for supplying the information to the computer; and

a user interface (in particular, a monitor) for receiving data from the computer in order to provide information to the user, wherein the received data are generated by the computer on the basis of the results of the processing performed by the computer.

A more detailed description of such a navigation system for computer-assisted surgery is the following. This navigation system preferably comprises the aforementioned computer for processing the data provided in accordance with the data processing method as described in any one of the preceding embodiments. The navigation system preferably comprises a detection device for detecting the position of the detection points which represent the main points and auxiliary points, in order to generate detection signals and to supply the generated detection signals to the computer such that the computer can determine the absolute main point data and absolute auxiliary point data on the basis of the detection signals received. In this way, the absolute point data can be provided to the computer. The navigation system also preferably comprises a user interface for receiving the calculation results from the computer. The user interface provides the received data to the user as information. Examples of a user interface include a monitor or a loudspeaker. The user interface can use any kind of indication signal (for example a visual signal, an audio signal and/or a vibration signal).

Within the framework of the invention, computer program elements can be embodied by hardware and/or software (this includes firmware, resident software, micro-code, etc.). Within the framework of the invention, computer program elements can take the form of a computer program product which can be embodied by a computer-usable, in particular computer-readable data storage medium comprising computer-usable, in particular computer-readable program instructions, “code” or a “computer program” embodied in said data storage medium for use on or in connection with the instruction-executing system. Such a system can be a computer; a computer can be a data processing device comprising means for executing the computer program elements and/or the program in accordance with the invention, in particular a data processing device comprising a digital processor (central processing unit—CPU) which executes the computer program elements and optionally a volatile memory (in particular, a random access memory—RAM) for storing data used for and/or produced by executing the computer program elements. Within the framework of the present invention, a computer-usable, in particular computer-readable data storage medium can be any data storage medium which can include, store, communicate, propagate or transport the program for use on or in connection with the instruction-executing system, apparatus or device. The computer-usable, in particular computer-readable data storage medium can for example be, but is not limited to, an electronic, magnetic, optical, electromagnetic, infrared or semiconductor system, apparatus or device or a medium of propagation such as for example the Internet. The computer-usable or computer-readable data storage medium could even for example be paper or another suitable medium onto which the program is printed, since the program could be electronically captured, for example by optically scanning the paper or other suitable medium, and then compiled, interpreted or otherwise processed in a suitable manner. Preferably, the data storage medium is a non-volatile data storage medium. The computer program product and any software and/or hardware described here form the various means for performing the functions of the invention in the example embodiments. The computer and/or data processing device can in particular include a guidance information device which includes means for outputting guidance information. The guidance information can be outputted, for example to a user, visually by a visual indicating means (for example, a monitor and/or a lamp) and/or acoustically by an acoustic indicating means (for example, a loudspeaker and/or a digital speech output device) and/or tactilely by a tactile indicating means (for example, a vibrating element or vibration element incorporated into an instrument).

Preferably, the inventive method is at least partly executed by a computer. That is, all steps or just some of the steps (i.e. less than a total number of steps) of the inventive method may be executed by a computer.

The expression “acquiring data” encompasses in particular (within the framework of a data processing method) the scenario in which the data are determined by the data processing method or program. Determining data in particular encompasses measuring physical quantities and transforming the measured values into in particular digital data and/or computing the data by means of a computer, in particular computing the data within the method of the invention. The meaning of “acquiring data” in particular also encompasses the scenario in which the data are received or retrieved by the data processing method or program, for example from another program, a previous method step or a data storage medium, in particular for further processing by the data processing method or program. Thus, “acquiring data” can also for example mean waiting to receive data and/or receiving the data. The received data can for example be inputted via an interface. “Acquiring data” can also mean that the data processing method or program performs steps in order to (actively) receive or retrieve the data from a data source, for instance a data storage medium (such as for example a ROM, RAM, database, hard disc, etc.) or via the interface (for instance, from another computer or a network). The data can achieve the state of being “ready for use” by performing an additional step before the acquiring step. In accordance with this additional step, the data are generated in order to be acquired. The data are in particular detected or captured (for example, by an analytical device). Alternatively or additionally, the data are inputted in accordance with the additional step, for instance via interfaces. The data generated can in particular be inputted (for instance, into the computer). In accordance with the additional step (which precedes the acquiring step), the data can also be provided by performing the additional step of storing the data in a data storage medium (such as for example a ROM, RAM, CD and/or hard drive), such that they are ready for use within the framework of the method or program in accordance with the invention. Thus, “acquiring data” can also involve commanding a device to obtain and/or provide the data to be acquired. The acquiring step in particular does not involve an invasive step which would represent a substantial physical interference with the body requiring professional medical expertise to be carried out and entailing a substantial health risk even when carried out with the required professional care and expertise. Acquiring, in particular determining, data in particular does not involve a surgical step and in particular does not involve a step of treating a human or animal body using surgery or therapy. This also applies in particular to any steps directed to determining data. In order to distinguish the different data used by the present method, the data are denoted (i.e. referred to) as “XY data” and the like and are defined by the information which they describe which is preferably called “XY information”.

In the following, example embodiments of the present invention are described with reference to the figures, which are merely to be regarded as examples of the invention without limiting the invention to the specific embodiment, wherein:

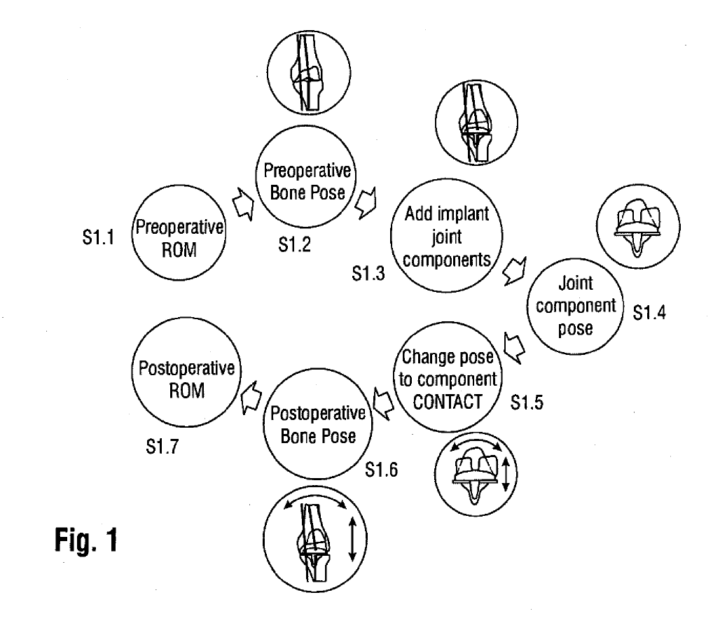

FIG. 1 illustrates the steps of the disclosed method for determining a post-operative pose of a joint;

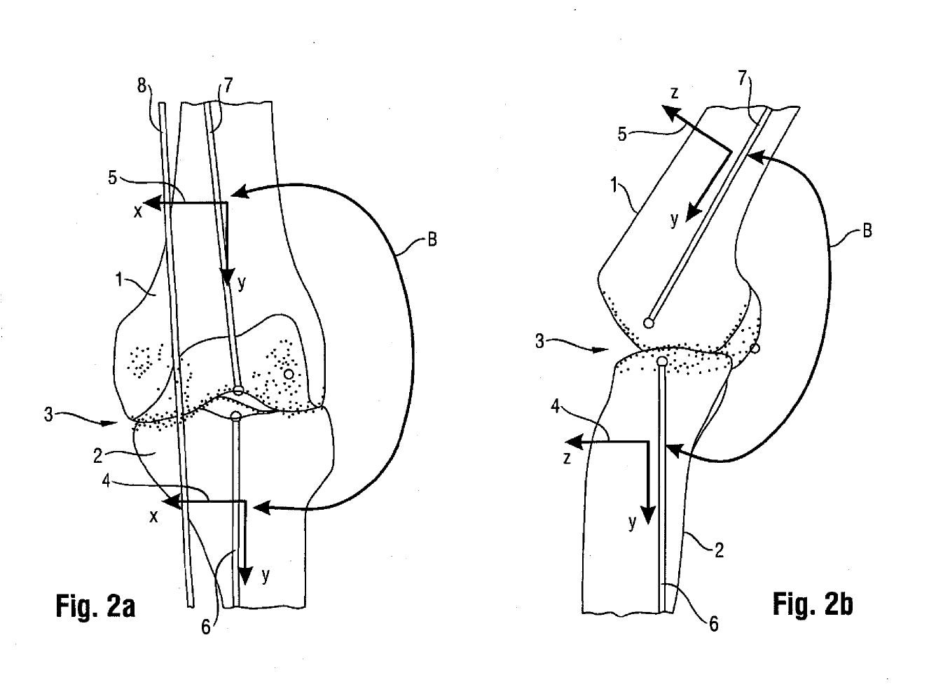

FIGS. 2a and 2b illustrate a single pose of the joint when viewed from two directions which are perpendicular to one another as well as the meaning of the initial body part position transformation;

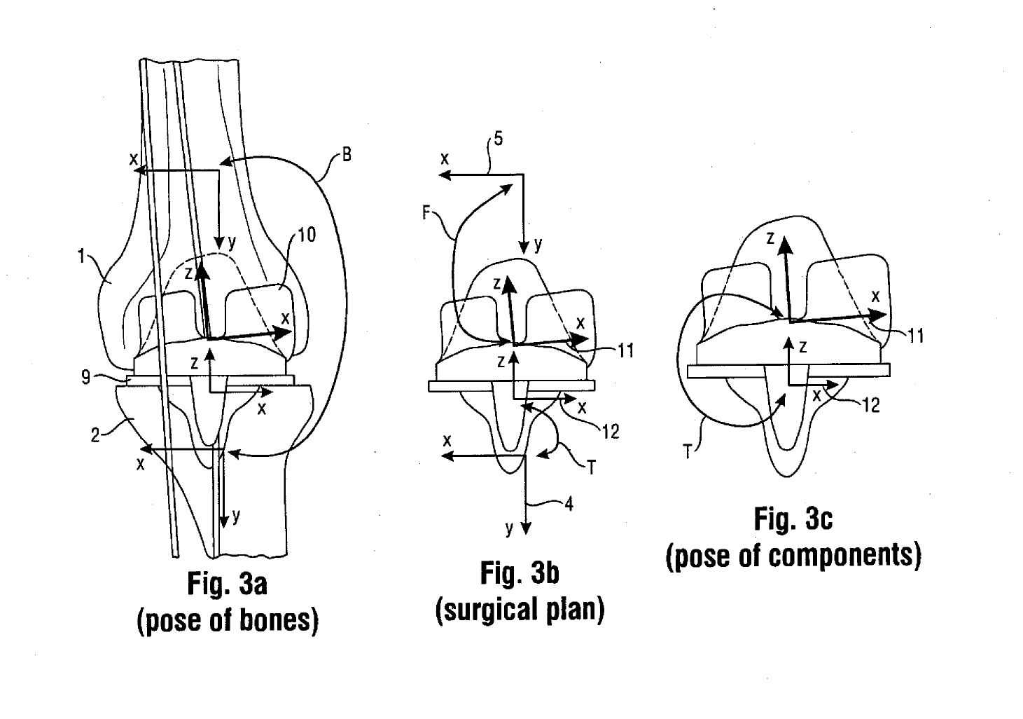

FIGS. 3a to 3c illustrate the meaning of the alteration transformation;

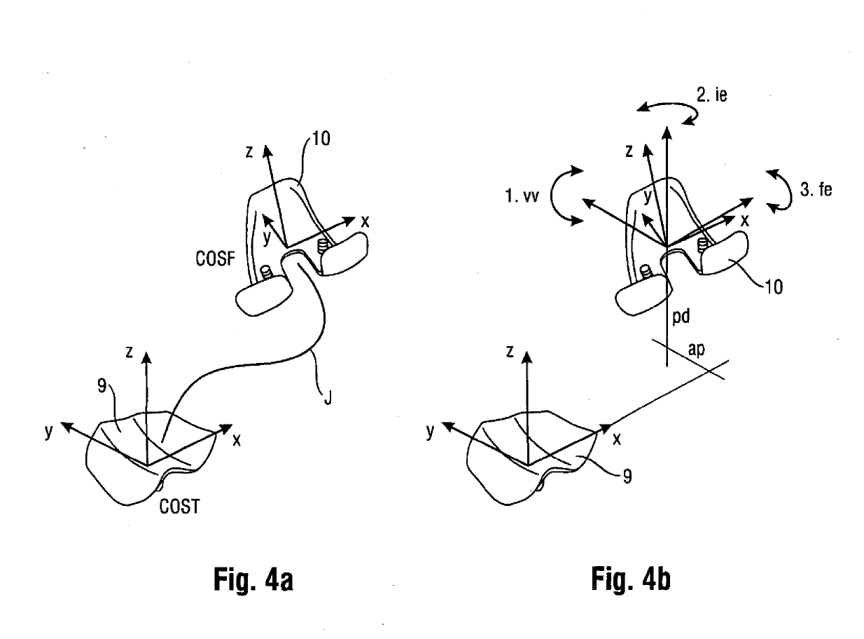

FIGS. 4a and 4b illustrate shifts and rotations for a transformation between the position of the first alteration part and the position of the second alteration part, which comprises an illustration for the altered body transformation information

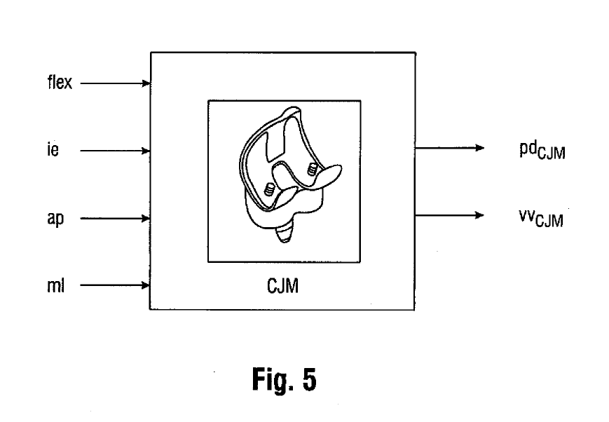

FIG. 5 illustrates the contact position parameters for given degrees of freedom of the first alteration part (femur implant component) with respect to a second alteration part (tibia implant component) derived from a continuous joint model;

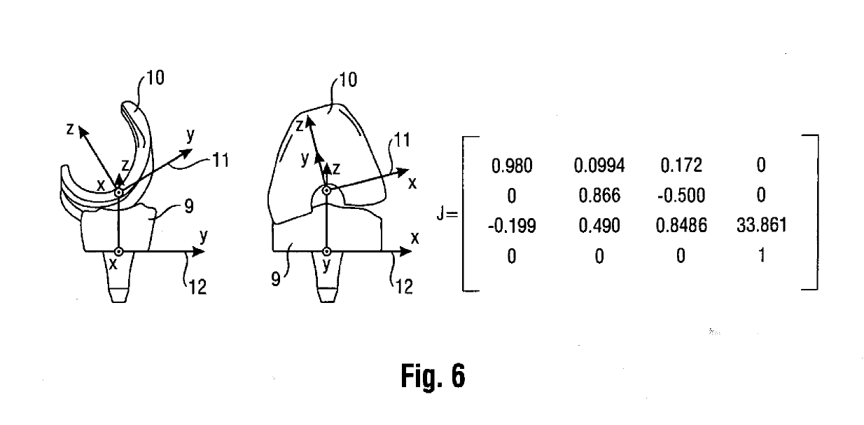

FIG. 6 illustrates a pose of the first and second alteration parts and a matrix representing a transformation between the positions of the two without the first and second alteration parts having the desired contact;

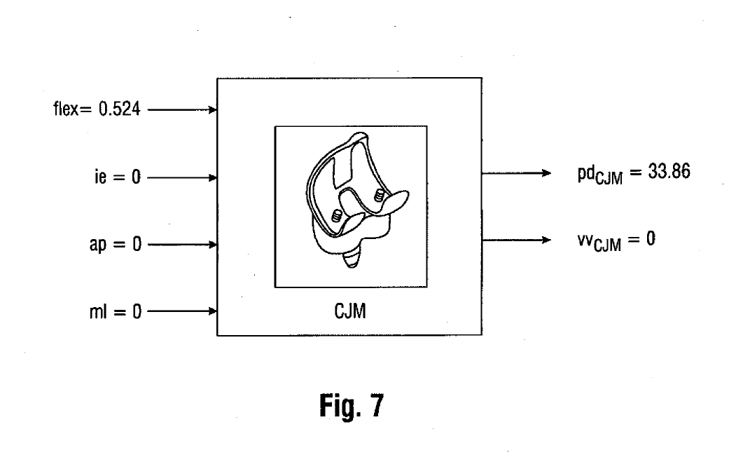

FIG. 7 illustrates the input of values into the continuous joint model for establishing a contact position, i.e. a position in which the first and second alteration parts have the desired contact with each other;

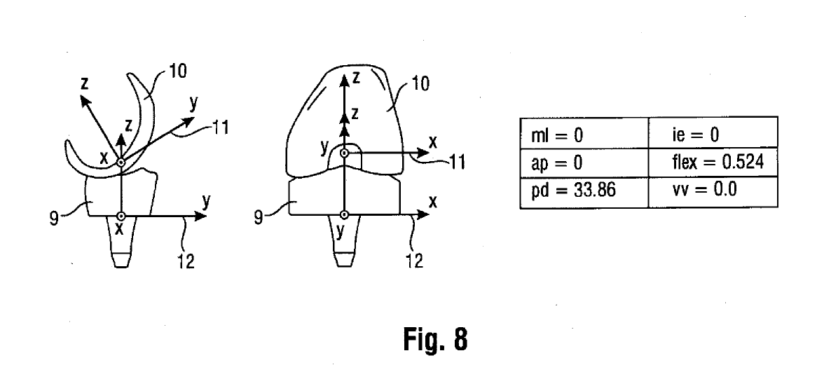

FIG. 8 illustrates a contact position of the first and second alteration parts derived from a continuous joint model for specific values of the parameters characterizing the degrees of freedom;

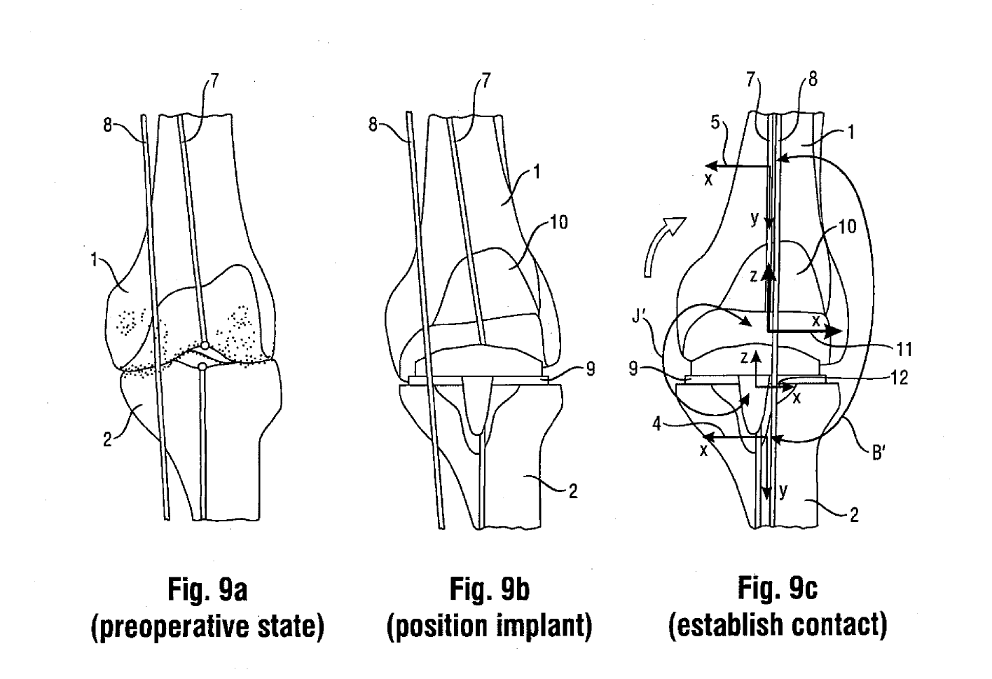

FIGS. 9a to 9c illustrate an adaptation of the pose of the joint with parameters generated by the continuous joint model;

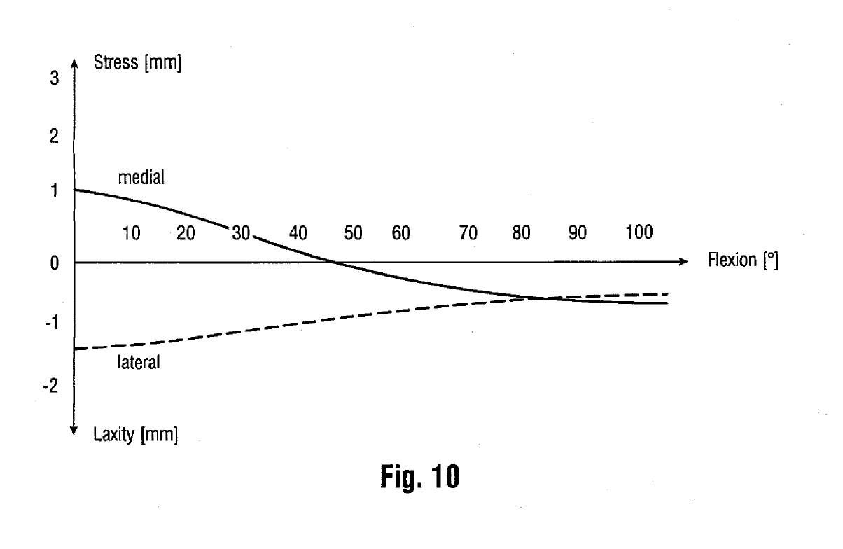

FIG. 10 illustrates an exemplary diagram of a positional shift of epicondyle points as a function of knee flexion;

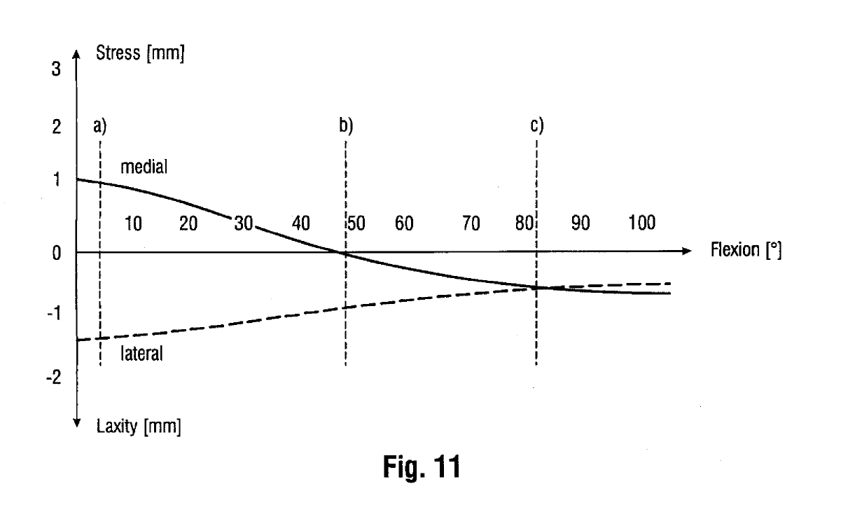

FIG. 11 illustrates the diagram of FIG. 10 and contains markings for regions of specific varus/valgus orientation of the knee;

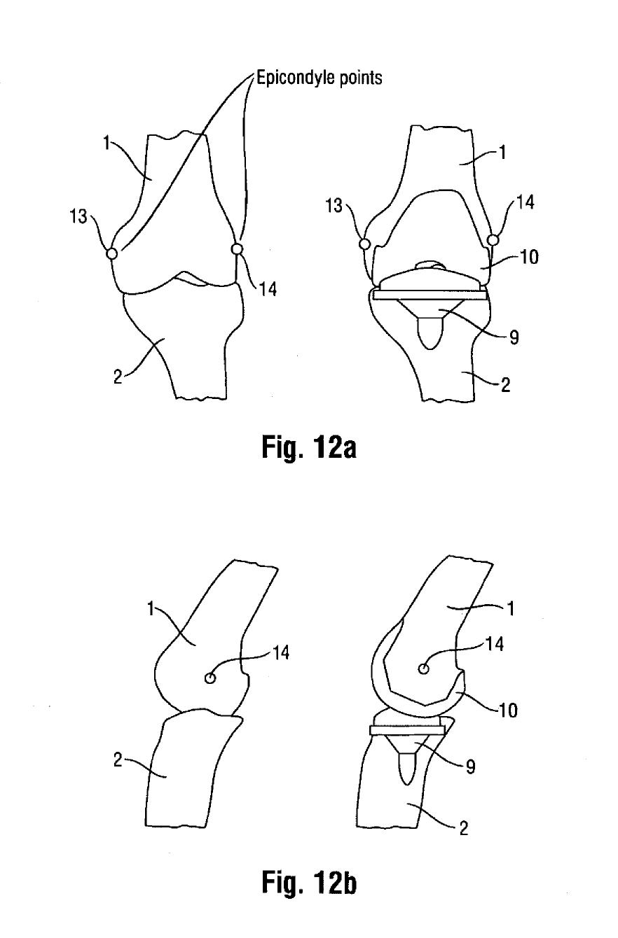

FIG. 12 illustrates live comparison views of the knee in the pre-operative state and the post-operative state in the frontal plane (FIG. 12a) and the sagittal plane (FIG. 12b);

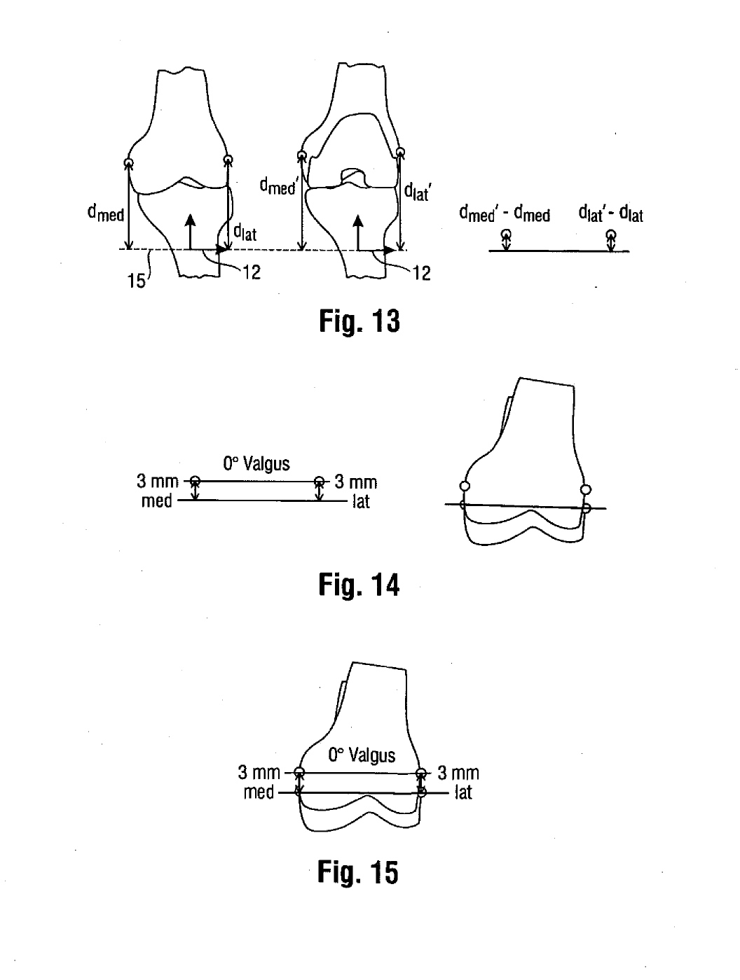

FIG. 13 shows a visualisation of a change of the distance of the epicondyle points from a predetermined position;

FIG. 14 is a visualisation of a pose change;

FIG. 15 is a graphical overlay of an outline of the first body part and the pose change diagram;

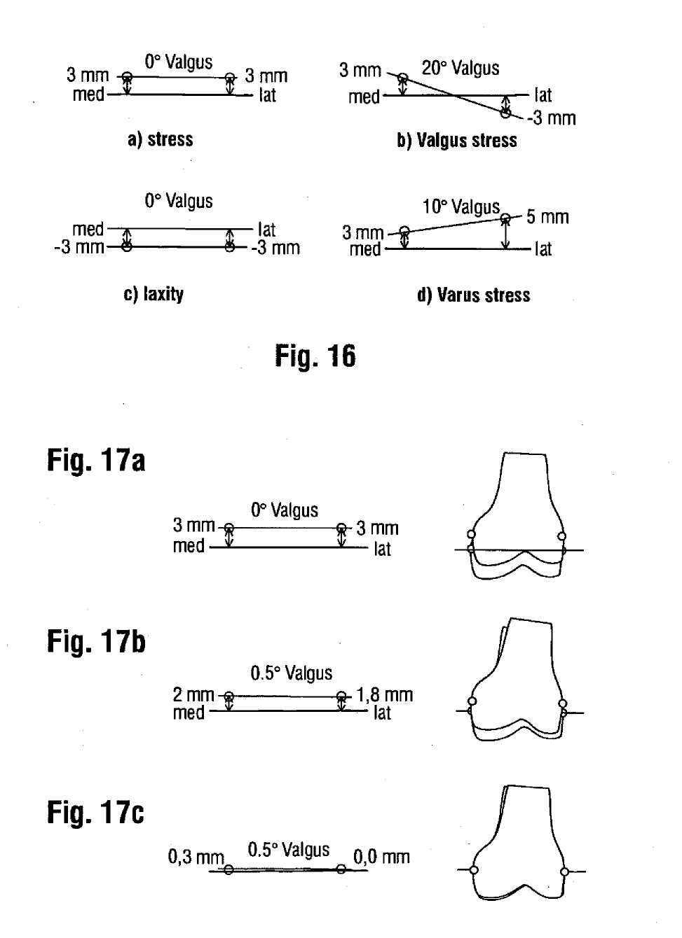

FIG. 16 illustrates stress patterns and pose changes for different varus/valgus angles;

FIG. 17 illustrates the stress check of a critical pose

A first embodiment of the present invention is directed to a procedure for adapting a pre-operatively acquired range of motion of an anatomical joint towards a post-operatively expected range of motion of that joint. In step S1.1 of FIG. 1, a pre-operative range of motion of the joint is acquired for example based on medical image data or position data acquired by an imaging apparatus or a navigation system in a pre-operative state of the joint. It consists of a series or sequence of specific movement states, in particular specific poses, of the joint. According to step S1.2, a dedicated bone pose in the pre-operative state is determined by extracting the initial body part position transformation information for two body parts which are connected by the joint from the medical image data or the position data acquired by the navigation system. In the case of FIG. 1, the joint is a knee and the body parts are the distal end of the femur and the proximal end of the tibia and the initial body part position transformation information is the bone pose. In step S1.3, first and second virtual anatomical alterations are implemented with regard to the body parts by adding data comprising alteration transformation information about the position and the geometry of femur-side and tibia-side implants for a total endoprosthesis of the knee (also called joint components)

Then, in step S1.4, altered body position transformation information (the pose of the implants), i.e. in particular a position and orientation of the implants (also called joint components) relative to one another, is determined based on the information about the pre-operative pose of the body parts. This pose of the implants is also termed joint component pose. The joint component pose in particular is a pose of the implants which is received when fitting the implants in their desired or necessary position relative to the respective body part to which they are to be attached. However, this may lead to a joint component pose in which the implants do not contact one another (in particular, do not contact each other in a predetermined, more particularly desired, manner). Therefore, step S1.5 continues with varying the altered body position transformation information (that is the joint component pose) such that the implants come into contact to one another. During this variation, the alteration transformation information (that is the relative position of each implant relative to the body part to which it is attached) is preferably kept constant. This allows to determine contact position information (this is a post-operative pose of the body parts comprising the implants in which the implants have sufficient contact with one another) (cf. step S1.6). This allows to determine in step S1.7 a post-operative range of motion of the body parts or the joint, respectively. The resulting post-operative range of motion then is a range of motion of the joint of which a stable contact of the implants with each other and is acquired based on the patient's natural and unaltered joint. The data which is received in particular step S1.7 allows for a straight forward graphical representation of the range of motion in both the pre-operative state and the post-operative state. Further, the received information allows for numerical analysis and identification of poses with greatest change of length and varus-valgus angle between the pre-operative state and the post-operative state. In order to determine the joint behaviour, a continuous joint model (CJM) is applied.

FIGS. 2a and 2b illustrate determination of a pre-operative bone pose, i.e. a pose of the body parts 1, 2 connected by a joint 3 in the pre-operative state. A three-dimensional, preferably rectangular first body part coordinate system 5 is assigned to the first body part 1. The first body part 1 rests in the first body part coordinate system 5. In analogy, a three-dimensional, preferably rectangular second body part coordinate system 4 is assigned to the second body part two. The second body part 2 rests relative to the second part coordinate system 4. FIG. 2a also illustrates the longitudinal axes 6, 7 of the first and second body parts 1, 2 (the tibia mechanical axis 6, and the femur mechanical axis 7) in a frontal view. FIG. 2b illustrates the features shown in FIG. 2a in a sagittal view. The illustrated pre-operative pose is defined by physical contact between the first and second body parts 1, 2 at the condyli of the femur representing the first body part 1 and of the tibia representing the second body part 2, as well as by a specific angle between the longitudinal axes 6, 7 which in this case involves both a varus/valgus angle between the longitudinal axis 7 of the femur and the proximal-distal (pd) direction 8 of the patient's body as well as a flexion angle between the longitudinal axis 6 of the tibia and the longitudinal axis 7 of the femur. The pre-operative pose is defined by the initial body part position transformation B which is acquired based on the positional information received from the medical image data or the navigation system. The transformation B describes a transformation between the positions of the first and second body parts 1, 2 in particular by comprising, more particularly consisting of a coordinate transformation between the first body part coordinate system 5 and the second body part coordinate system 4.

FIG. 3a illustrates the addition of implant joint components according to step S1.3 which represents the first and second virtual anatomical alteration. The first virtual anatomical alteration is directed to fitting an implant (a first alteration part 10) to the first body part 1 represented by the femur, the second virtual anatomical alteration is directed to fitting a second, other implant (a second alteration part 9) to the second body part 2 represented by the tibia. The position of the first implant 10 relative to the first body part 1 is defined by a surgical plan, in particular by way of a coordinate transformation F between the first body part coordinate system 5 and a first alteration part coordinate system 11 (assigned to the first alteration part 10). The first alteration part 10 rests relative to the first alteration part coordinate system 11. The position of the second alteration part 9 relative to the second body part 2 is also defined by the surgical plan, in particular by way of a coordinate transformation T between the second body part coordinate system 4 and the second alteration part coordinate system 12 (assigned to the second alteration part 9). The second alteration part 9 rests relative to the second alteration part coordinate system 12. Such is also illustrated by FIG. 3b.

One aim of the disclosed method is to determine a transformation J between the first alteration part coordinate system 11 and the second alteration part coordinate system 12. The transformation J may be determined by the equation J=F·B·T⁻¹, where J, F, B and T are defined as 4×4 matrices.

The information about the entries (in particular, the entries themselves and/or their values) of F and T represent the alteration transformation information and is contained in the surgical plan representing the alteration transformation data. The translational (shifting) and rotational (angular) degrees of freedom of the first and second alteration parts 10, 9 are illustrated by FIGS. 4a and 4b. In particular, the entries of the matrix J are defined as follows: The shift ml (in the x-direction of the second body part coordinate system 4), the ap-direction (in the y-direction of the second body part coordinate system 4) and the pd-direction (in the z-direction of the second body part coordinate system 4) correspond to the elements of transformation matrix J as follows: ml=J₁₄; ap=J₂₄; pd=J₃₄.

The rotations follow the sequence vv (the first rotation around the y-direction of the second body part coordinate system 4), ie (the second rotation around the z-axis of the second body part coordinate system 4), flex (the third rotation around the x-axis of the second body part coordinate system 4).

For varying the joint component pose to produce the predetermined (desired) physical contact between the components, a sequence of rotations is conducted as follows. At the beginning of the sequence, the femur implant coordinate system COSF representing the first body part coordinate system 5 is aligned with the tibia coordinate system COST representing the second body part coordinate system 4. The first step is a rotation of COSF around the y-axis of COST by an angle vv (all angles hereinforth being defined in radians). As a result, COSF changes its orientation. The next step is a rotation of COSF around its moved z-axis by an angle ie. The last step is a rotation of COSF around its new x-axis by an angle flex. The second orientations are applied to coordinate system axis moved by a subsequent rotation. From the elements of transformation matrix T, the rotations can be derived as follows:
ie=arcsin(J₂₁);
flex=arcos(J₂₂/cos(ie)) for J₂₃<0, flex=−arccos(J₂₂/cos(ie)) for J₂₃>=0;
vv=arcsin(J₃₁/cos(ie)).

The derived parameters ml, ap, ie, pd, flex and vv give a complete and equivalent description of the position of the first and second alteration parts 10, 9 relative to one another as defined by J.

The pose described by the derived parameters will not necessarily describe a valid pose of the alteration parts which is intended by their manufacturer, in particular a pose in which the predetermined (desired) contact is established. For example, the alteration parts may intersect each other or have no contact at all, since their positions relative to the respective first or second body part, in particular relative to the bone stems of the femur and the tibia, are acquired from the surgical plan and their relative position with respect to each other is acquired based on the pre-operative range of motion, i.e. the initial body part transformation information. The respective positions may therefore not match to establish the predetermined contact. In a next step S1.5 (cf. FIG. 1), the pose of the alteration parts is varied in order to establish contact between the alteration parts. For the purpose of establishing the predetermined contact, the derived parameters ml, ap, ie and flex are fed into a continuous joint model (CJM) which will generate output parameters pd_{CJM} and vv_{CJM} which support the predetermined (desired), in particular stable and intended contact state of the alteration parts, in particular implant and or/joint components. FIG. 5 is a visual representation of the input and output parameters for the CJM. For at least the aforementioned reasons, the output parameters pd_{CJM} and vv_{CJM} will most likely deviate from the parameters pd and vv which are found by the composition of the transformation matrix J.

An example pose of the first alteration part 10 and the second alteration part 9 together with an orientation of the first alteration part coordinate system 11 and the second alteration part coordinate system 12 is shown in FIG. 6 along with an example of the transformation matrix J. The pose shown in FIG. 6 is a pose in which no contact in the sense of the aforementioned definition of predetermined (desired) contact is established between the first alteration part 10 and the second alteration part 9. The decomposition of the transformation matrix J into parameters yields in accordance with FIG. 6: ml=0; ap=0; pd=33.86; ie=0; flex=0.524; vv=0.2.

FIG. 7 describes the procedure of feeding ml, ap, ie and flex into the CJM, whereby values for pd_{CJM} and vv_{CJM} are established and support, in particular ensure the predetermined contact between the first alteration part 10 and the second alteration part 9. The pose of the alteration parts 9, 10 resulting from these parameters is shown in FIG. 8. In the case of FIG. 8, vv is set to vv_{CJM}=0.0 and pd is set to pd_{CJM}=33.86. In this example case, pd and pd_{CJM} are equal, but their rotation parameter vv deviates from vv_{CJM} by 0.2 radians (equivalent to 11.5°). FIG. 8 shows a pose of the alteration parts 9, 10 in which they have the predetermined (desired), in particular stable physical contact.

FIGS. 6 and 8 show poses of the first alteration part 10 and the second alteration part 9 represented by a femur-side implant 10 and a tibia-side implant 9 for a knee total endoprosthesis (TEP) which are taken along directions which are perpendicular to each other. The respective left representation is in a sagittal view and the respective right representation is in a frontal view.

FIG. 9 describes adapting the pose of the first body part 1 and second body part 2 which is acquired when the first alteration part 10 and the second alteration part 9 are fitted to the first body part 1 and the second body part 2, respectively, by way of first and second virtual anatomical alterations based on a surgical plan as shown in detail by FIG. 3b and also by FIG. 9b showing only the resulting implant positions. Compared to the bone pose defined by the pre-operative state shown in FIG. 9a, no predetermined contact is established between the first body part 1 and the second body part 2 or the first alteration part 10 and the second alteration part 9, respectively. In contrast thereto, as can be seen from FIG. 9a, the first body part 1 and the second body part 2 are in predetermined contact in the pre-operative state. The method as described above allows to determine necessary changes to the bone pose of FIG. 9b in order to establish the predetermined (desired) contact between the first alteration part 10 and the second alteration part 9 or the first body part 1 and the second body part 2, respectively, as depicted by FIG. 9c. As shown in FIG. 9c, contact position information consists of an adapted body part transformation B′ between the first body part coordinate system 5 and the second body part coordinate system 4 and B′ is determined according to the following equation:

B′=F⁻¹·J′·T

In this equation, the transformation matrix J′ describes the spatial relation, in particular transformation between the position of the first alteration part 10 (in this case a femur-side implant) and the position of the second alteration part 9 (in this case a tibia-side implant) which now have the predetermined, in particular stable physical contact in the pose of FIG. 9c, wherein J′ is determined from the parameters ml, ap, ie, flex, vv_{CJM} and pd_{CJM} as follows:

J′=Transₓ(ml)·Trans_{y}(ap)·Trans_{z}(pd_{CJM})·Rot_{y}(vv_{CJM})·Rot_{z}(ie)·Rotₓ(flex)

Transₓ(ml) is a simple translation transformation describing a shift along the x-axis of the second alteration part coordinate system 12 by a value of ml. Trans_{y}(ap) and Trans_{z}(pd_{CJM}) describe shifts along the y- and z-axes of the second alteration part coordinate system 12 after undergoing the transformation by Transₓ(ml) and Trans_{y}(ap), respectively. After the shifts have been applied, standard rotation matrices follow. Rot_{y}(vv_{CJM}) defines a rotation around the y-axis of the transformed second alteration part coordinate system 12 after undergoing each of the preceding transformations in the given order by the adapted varus/valgus angle vv_{CJM}. Rot_{z}(ie) defines a rotation around the z-axis of the transformed second alteration part coordinate system 12 after undergoing each of the preceding transformations in the given order by the initial ie value. Rotₓ(flex) defines a rotation around the x-axis of the transformed second alteration part coordinate system 12 after undergoing each of the preceding transformations in the given order by the initial flex value.

The actual postoperative poses will not only require the alteration parts to have the predetermined contact. At the same time each pose will result from the interaction of ligaments stabilizing the joint. It will remain to some level uncertain, in which direction the ligaments will change the predicted towards the actual parameters of the postoperative pose. For example, the postoperative joint might become instable for too much laxity being introduced by a considerable decrease of proximodistal shift. The predicted pose would then be compromised by growing freedom of the joint to shift in the transversal plane (detected in the real knee when applying Lachman- and Drawer-Tests). On the contrary, an increase of proximodistal shift would stress the ligaments and widen the joint gap. Some unforeseen amount of posterior shift could be introduced by the ligaments to take advantage of the tibial posterior slope and narrow the gap.

In order to acquire information about the contact enforced deviation that induces i.e. (mechanical) stress or laxity in the ligaments, the following further method steps are described which may combine with the aforementioned features of the method of adapting the pose. However, these following further steps may also serve as a self-contained method of determining contact enforced deviation of a joint without requiring the aforementioned method features.

The aforementioned adaption of a joint pose in the pre-operative state towards a corresponding joint pose in the post-operative state provides valuable information for a user such as a surgeon about stress conditions in the joint, in particular about stress conditions of elastic constituents of the joint such as connecting structures like ligaments and/or cartilage. A set of techniques for analysing and visualizing an adapted pose or a complete range of motion is provided, wherein the adapted pose is determined and preferably graphically output immediately after the pose in the pre-operative state has been acquired. This visualisation technique works online or, as it is often called, constitutes a live comparison technique for comparing the pose of a joint, in particular in the joint, in the pre-operative state and a pose in the (predicted) post-operative state. This comparison is supported by visualising views of the body parts, in particular bones which are connected by this joint, such as the tibia and the femur in the case of the joint being a knee. According to a specific embodiment of this technique, predetermined locations on the first body part 9 such as the epicondyle points (cf. FIGS. 12a and 12b) are determined and a pose change for the joint in the post-operative state is determined based on a change of the distance of the epicondyle points from a preferably common baseline. The pose change is then visualised based on the determined difference in distances. A specific embodiment of this technique is directed to a live stress check for critical poses in the post-operative state, wherein the results of the live stress check are fed back into the diagram of FIG. 10 in order to create the diagram of FIG. 11.

FIG. 10 illustrates the dependence of the difference in distance of the medial epicondyle point (continuous curve in FIG. 10) and lateral epicondyle point (dashed curve in FIG. 10) of a knee joint from a predetermined position , in particular a common reference line from which the distance of both epicondyle points is measured, in dependence on the flexion angle of the knee joint. The difference is preferably determined by subtracting the distance in the pre-operative state from the distance in the post-operative state so that a negative result of the difference indicates a laxity in the joint in the post-operative state, whereas a positive result of the difference indicates a stress in the post-operative state compared to the pre-operative state. An intuitive and straightforward way to present the consequences of the pose adaption driven by the inserted joint components is a live view of the preoperative and the adapted postoperative pose in comparison to each other as shown in FIGS. 12a and 12b.

FIG. 12a illustrates a frontal view of a knee joint represented by a first body part (femur) 1 and a second body part (tibia) 2 along with the lateral epicondyle point 14 and the medial epicondyle point 13. The left-side representation in FIG. 12a shows the pre-operative state of the knee, the right representation of FIG. 12a shows the post-operative state of the knee including the first alteration part (femur-side implant) 10 and the second alteration part (tibia-side implants) 9. FIG. 12b shows on the left side a view of the knee in the sagittal plane in a lateral viewing direction and on the right side the analogous view of the knee in the post-operative state. The representations of the post-operative state in FIGS. 12a and 12b show the adapted pose with the implants 9, 10 in physical contact with each other, and the accordingly adapted bone pose of tibia and femur. The epicondyle points are highlighted in the graphical display to give the user a quick reference for checking the consequences of the pose adaption at first glance. If the epicondyle points move to proximal, ligament tension is increased. If they move distally, laxity can be introduced which may be done on purpose or be an undesired side-effect. Since the adaption of the pose can be achieved on the fly (i.e. online), the adapted views can be shown on-line during the acquisition of the initial body part position transformation data. Pose change visualization focuses on the changes during the pose adaption and is shown in FIGS. 14, 15, 16 and 17.

To give the user quantitative information about the pose adaption, the position of the epicondyle points is determined in the pre-operative state and the post-operative state and compared between its states. The epicondyle points are close to the femoral origins of the collateral ligaments, and their position in the post-operative state, in particular relative to the tibia including any possible deviation of this position is an indicator for a change in ligament tension in the joint between the pre-operative state and the post-operative state. If the medial epicondylar point moves to proximal, the inserted implants 9, 10 open up the joint gap on this side and the tension of the medial ligament (i.e. of the medial ligament fibers) is increased. If the medial epicondylar point moves distally, the tension is decreased and the ligament(s) on the medial side can experience laxity. The same applies to the lateral epicondyle point and the ligament(s) on the lateral side. However, if the two epicondylar points change their position in contrarian directions (in particular in the proximal-distal direction), a varus or valgus rotation (i.e. a varus or valgus angular movement) is introduced into the joint. This can increase stress on one side of the knee and the respective collateral ligament and increase laxity (i.e. decrease stress) on the other side of the knee and the respective collateral ligament.

In order to present quantitative information about any changes in stress and/or laxity in the joint (all its ligaments, respectively), the positional deviation of the epicondyle points between the pre-operative state and the post-operative state is determined as explained by FIGS. 13 and 14. The distance d_{med} of the medial epicondylar point to a predetermined position 15 (in this case, the a dedicated transversal plane of the second body part (tibia) coordinate system 12) is measured in the pre-operative state. The corresponding distance d′_{med} is determined in the post-operative state. In analogy, the distance dₗₐₜ of the lateral epicondyle point from the predetermined position 15 is determined in the pre-operative state and as d′ₗₐₜ in the post-operative state. For both pairs of distances, a difference is calculated by subtracting d_{med} from d′_{med} (d′_{med}−d_{med}) and by subtracting dₗₐₜ from d′ₗₐₜ (d′ₗₐₜ+dₗₐₜ).

The difference between the preoperative and the adapted position in proximodistal direction of each point (e.g. d_{med}′−d_{med}) is presented as a distance off a common bottom line (see FIG. 13 on the right and FIG. 14 on the left). The bottom line represents the preoperative position of the epicondyle points. For good visualization, it stays fixed for all poses and represents a common preoperative level. The positions of the two balls represent the calculated distances of both epicondyle points from their preoperative position (the bottom line). These distances will most likely vary for each pose. Additionally or alternatively, the varus/valgus angle, the leg length (or change in leg length) or changes in the values of the parameters ml, ap, pd, flex, ie, vv may be determined based on the altered body part position transformation data in order to describe a change in pose from the pre-operative state to the post-operative state.

FIG. 14 shows that if both differences have the same value (in this case, 3 mm), a valgus angle of 0° is achieved. On the right side of FIG. 14, a comparison of the femur pose in the pre-operative state (light shading) and the post-operative state (dark shading) is given while the top horizontal line through the epicondyle points on the left side of FIG. 14 indicates that the epicondyle points in the postoperative state have moved to proximal.

FIG. 15 shows that the proximal shift of the position of the epicondyle points is equal for the medial and the lateral side and therefore no additional varus/valgus rotation of the femur occurs in post-operative state with regard to the preoperative state.

FIG. 16 provides examples of stress (FIG. 16a) or laxity (FIG. 16c) on the joint (or ligaments, in particular collateral ligaments) at 0° valgus angle in the post-operative state. Furthermore, a valgus stress at 20° valgus angle due to shifting of the epicondyle points to contrarian directions in the post-operative state is depicted (FIG. 16b). FIG. 16d shows a condition of varus stress in the post-operative state due to a shifting of the lateral epicondyle point by a distance of 5 mm which is larger than a shifting of the medial epicondyle point by a distance of 3 mm. The shifting of the epicondyle points according to FIG. 16d occurs in the same direction, in this case in the proximal direction, however by different amounts of shifting which leads to an uneven distribution of stress among the medial and lateral collateral ligaments and a resulting varus stress component on the joint.

The presentation of the change of contact enforced deviation in the joint can be made even more intuitive for a user by showing or superimposing the femoral bone outline onto the diagram (cf. FIGS. 14 and 15).

Predetermined locations on the first body part 10 and the second body part 9 other than the epicondyle points can be used as a reference, too. For example, contact points of the natural joint (i.e. the joint in the pre-operative state) or other characteristic points fixed to the bones, for example in the regions of the anterior cortex or in the fossa intercondylaris. However, the epicondyle points provide an easily comprehensible graphic and descriptive reference with suitable neighbourhood to the ligament origins.

FIG. 17 shows the principles of a stress check for critical poses. It is to be noted that the virtual anatomical alterations, in particular implant insertions, change the kinematic, i.e. moving behaviour, of the joint. For example, poses that are easily applied in the pre-operative state may be unreachable in the post-operative state. Under adverse conditions, a post-operative extension of flexion deficit may result when full extension of flexion poses introduce very high ligament tension that block the joint. On the contrary, laxity can introduce instability into the joint. A lack of ligament tension may allow the joint to engage at the different anterioposterior positions for the same flexion value whereas a healthy joint would not show any tolerance for such shift. The commonly known Drawer- and Lachman-Tests exploit this behavior in orthopedic surgery.

The occurrence of such conditions can be simulated before surgery takes place with a stress check utilizing the aforementioned visualisation techniques and principles. For a critical pose (for example at 90° flexion angle or at any other flexion angle) under check, the surgeon will apply manual stress to the joint while the initial body part position transformation data is being acquired. In this manner, information about the behaviour of the joint in the pre-operative state under stress conditions can be acquired. If this pose is then used as a basis for the virtual anatomical alterations, the behaviour of the joint in the same pose in the post-operative state can be determined In the graphical display, the pose in the pre-operative state and the pose in the post-operative state can be overlaid with utmost congruency. If this can be achieved by a tolerable amount of manually exerted stress, that specific pose has passed the stress test. It is then determined that this pose will work after surgery has been completed, i.e. with the implant components inserted, and the joint will not be blocked from attaining that pose. Overlaying the poses in the pre-operative state and the post-operative state can be supported by displaying the pose change diagram according to FIG. 10 or FIG. 11, augmented with the bone outline or assisted by the live comparison view according to in particular FIGS. 14 and 15.

For documenting the pose changes (in particular, the change in a varus/valgus angular orientation) of the joint, across a complete range of motion in the post-operative state, the distance differences of both epicondyle points are plotted in a diagram as a function of joint flexion (cf. FIG. 11). The curve shown in FIG. 11 are the same as those shown in FIG. 10. However, FIG. 11 additionally provides an indication of characteristic sections in the diagram which differ in stress conditions. For example, indication a) denotes a region of varus/valgus stress, indication b) denotes a region of passage into bilateral ligament laxity, and indication c) indicates a region of joint instability. When both curves are in the stress region (a situation as shown in the diagram of FIG. 10 or 11), the joint might block movement at such or to such poses. As an alternative to FIG. 11, the varus/valgus angle as calculated by in particular the method described with reference to FIGS. 13 to 15 can be drawn as one curve. A second curve will then be plotted as an average of the medial and the lateral distance differences. This alternative illustration offers the same basic information about level and direction of stress in the joint.

The method disclosed herein gives valuable hints to judge a post-operative outcome of an envisaged joint surgery, in particular knee surgery. For a pre-operatively stable joint, critical post-operative poses can be identified that would stress the ligaments after implant insertion: If the predicted proximal distal shift deviates from the pre-operative state, the cruciate ligaments experience stress or laxity. If the predicted varus/valgus angle deviates, the collateral ligaments suffer from stress or laxity. In this way, the predicted parameters can be used by a surgeon to identify critical post-operative poses and choose his surgical options accordingly.

The integration of the range of motion (ROM) adaption offers a variety of useful intelligence for the surgical planning procedure:
the prediction of post-operative movement shown by synthetic ROM animation of the joint,
the deviation of the pre-operative and post-operative bone movement given by numerical analysis of the main angles and shifts,
the impact of a change in the surgical plan (e.g. to implant component position and orientation) to the change in post-operative movement by same means,
the identification of critical post-operative bone poses, where a big deviation in shift or angle from the pre-operative pose occurs and possibly applies in excessive stress or laxity to the ligaments and compromises the joint situation.

In particular, if a numerical analysis shows regions in the range of motion with a strong deviation in varus/valgus angle or leg length from the pre-operative state, these regions can be made subject of a tension check in the real patient's joint. Using the given angles and shifts of any critical pose, the surgeon may adjust the patient's knee to such a pose with support of the navigation system and check the ligament tension in the associated pose, either manually by tactile feedback or with dedicated tension measurement devices. Based on the outcome, he will choose his options and stay with the current plan, opted for a change of the surgical plan or pursue a ligament release to prevent post-operative risks of excessive strain or laxity in the joint.

1. A data processing method for determining a position of at least two body parts relative to one another, which at least two body parts are connected to one another by at least one joint, the method being executed by a computer and comprising:
a) acquiring initial body part position transformation data comprising initial body part position transformation information describing an initial body part position transformation between positions of the at least two body parts in a predetermined pose of the at least two body parts;
b) acquiring alteration transformation data comprising alteration transformation information describing an alteration transformation between a position of a first alteration part of at least one first body part of the at least two body parts, which first alteration part represents a first virtual anatomical alteration to the at least one first body part, and a position of an anatomically unaltered part of the at least one first body part;
c) determining, based on the initial body part position transformation data and the alteration transformation data, altered body part position transformation data comprising altered body part position transformation information describing a transformation between a position of the first alteration part and a position of a second other one of the at least two body parts in the predetermined pose; and
d) determining, based on the altered body part position transformation data, contact position data comprising contact position information describing positions of the at least two body parts in which at least two of the at least two body parts are considered to have direct or indirect physical contact with one another, wherein the contact position data is determined based on acquiring a data set of parameters including: a mediolateral (ml) parameter, an anterioposterior (ap) parameter, a proximodistal (pd) parameter, an internal/external rotation (ie) angle parameter, a flexion angle parameter, and a vaus-valgus angle parameter, wherein the parameters describe the altered body part position transformation information, acquiring a data set comprising a subset of n of the parameters that includes at least one of the ml parameter, the ap parameter, the pd parameter, and the flexion parameter as an input parameter data set, wherein n is an integer in a range of one to five describing the number of predetermined parameters, determining a modelled 6-n free parameters based on proximodstal modelled values and varus-valgus modelled values of the altered body part position transformation information and changing the altered body part position transformation information according to the determined 6-n free parameters, and wherein a resulting pose of the first and second body parts is determined based on the contact position data, which contact position data is determined based on the changed altered body part position transformation data.

2. The method according to claim 1, wherein the alteration transformation information further describes a transformation between a position of a second alteration part of the second body part, which second alteration part represents a second virtual anatomical alteration to the second body part, and a position of an anatomically unaltered part of the second body part, and wherein the altered body part information describes a transformation between the position of the first alteration part and the position of the second alteration part.

3. The method according to claim 1, wherein the first virtual anatomic alterations comprises conducting a virtual implant, and wherein, if the alteration transformation information further describes a transformation between a position of a second alteration part of the second body part, which second alteration part represents a second virtual anatomical alteration to the second body part, and a position of an anatomically unaltered part of the second body part, and wherein the altered body part information describes a transformation between the position of the first alteration part and the position of the second alteration part, the second virtual anatomical alternation comprises conducting an implant.

4. The method according to claim 1, wherein the initial body part position transformation data is acquired by a navigation system.

5. (canceled)

6. (canceled)

7. The method according to claim 1, wherein the contact position information describes positions of the at least two body parts in which the first alteration part is considered to have direct or indirect physical contact with an unaltered part of another one of the at least two body parts or, if the alteration transformation information further describes a transformation between a position of a second alteration part of the second body part, which second alteration part represents a second virtual anatomical alteration to the second body part, and a position of an anatomically unaltered part of the second body part, and wherein the altered body part information describes a transformation between the position of the first alteration part and the position of the second alteration part, with another alteration part of another one of the at least two body parts.

8. The method according to claim 1, wherein a plurality of positions of at least two of the at least two body parts relative to one another in different functional poses is determined based on the altered body part position transformation information.

9. The method according to claim 8, comprising a step of determining, based on the altered body part position transformation data, expected contact enforced deviation data comprising expected contact enforced deviation information describing a predetermined relationship between an expected change of movement state, in particular an expected change of pose, of the at least two body parts due to the altered body part position transformation and a tension attribute, in particular at least one of stress and laxity, of the joint.

10. The method according to claim 9, wherein determining the expected contact enforced deviation data comprises comparing a distance of predetermined locations on the first body part from a predetermined position before the first virtual anatomical alteration to a distance of the predetermined locations from the predetermined position after the first virtual anatomical alteration or the second virtual anatomical alteration.

11. The method according to claim 10, wherein the predetermined position is a reference line or reference plane before and/or after application of the first virtual anatomical alteration or the second virtual anatomical alteration, wherein the reference line is in particular parallel to a transversal plane of a body of which the body parts are part of.

12. The method according to claim 10, wherein comparing the distances comprises determining a difference between the distances.

13. The method according to claim 10, comprising graphically displaying graphical representations of the at least two body parts in particular in a plurality of movement states, in particular in a plurality of poses, after the first virtual anatomical alteration has been implemented.

14. The method according to claim 10, wherein the joint is a knee joint and an angle, in particular a varus and/or valgus angle, between straight lines connecting the predetermined locations before and/or after the virtual anatomical alteration is determined based on the comparison of the distances.

15. A computer program which, when running on a computer or when loaded onto a computer, causes the computer to
a) acquire initial body part position transformation data comprising initial body part position transformation information describing an initial body part position transformation between positions of the at least two body parts in a predetermined pose of the at least two body parts;
b) acquire alteration transformation data comprising alteration transformation information describing an alteration transformation between a position of a first alteration part of at least one first body part of the at least two body parts, which first alteration part represents a first virtual anatomical alteration to the at least one first body part, and a position of an anatomically unaltered part of the at least one first body part;
c) determine, based on the initial body part position transformation data and the alteration transformation data, altered body part position transformation data comprising altered body part position transformation information describing a transformation between a position of the first alteration part and a position of a second other one of the at least two body parts in the predetermined pose; and
d) determine, based on the altered body part position transformation data, contact position data comprising contact position information describing positions of the at least two body parts in which at least two of the at least two body parts are considered to have direct or indirect physical contact with one another, wherein the contact position data is determined based on acquiring a data set of parameters including: a mediolateral (ml) parameter, an anterioposterior (ap) parameter, a proximodistal (pd) parameter, an internal/external (ie) rotation angle parameter, a flexion angle parameter, and a vaus-valgus (vv) angle parameter, wherein the parameters describe the altered body part position transformation information, acquiring a data set comprising a subset of n of the parameters that includes at least one of the ml parameter, the ap parameter, the pd parameter, and the flex parameter of n of the parameters in the data set as an input parameter data set, wherein n is an integer in a range of one to five describing the number of predetermined parameters, determining a modelled 6-n free parameters based on proximodstal modelled values and varus-valgus modelled values of the altered body part position transformation information and changing the altered body part position transformation information according to the determined 6-n free parameters, and wherein a resulting pose of the first and second body parts is determined based on the contact position data, which contact position data is determined based on the changed altered body part position transformation data.
